(19) United States
(12) Patent Application Publication
(10) Pub. No.: US 2019/0076289 A1
(43) Pub. Date: Mar. 14, 2019

(54) RING-INNER-DIAMETER-ADJUSTABLE MULTI-PROTRUSION RING PERINEAL REGION MUSCLE EXERCISE EQUIPMENT

(71) Applicant: Hyun Joon MOON, Gwangju (KR)

(72) Inventor: Hyun Joon MOON, Gwangju (KR)

(21) Appl. No.: 16/093,785

(22) PCT Filed: Apr. 19, 2017
(86) PCT No.: PCT/KR2017/004176

§ 371 Date: Oct. 15, 2018

(30) Foreign Application Priority Data
Apr. 29, 2016 (KR) 10-2016-0053361

(51) Int. Cl. A61F 5/41; A63B 23/20; A63B 21/00; A63B 21/02

(52) U.S. Cl. CPC A61F 5/41; A63B 23/20; A63B 21/00069; A61F 2005/414; A63B 21/4001; A63B 21/4025; A63B 21/02

(57) ABSTRACT

Provided is a ring-inner-diameter-adjustable multi-protrusion ring perineal region muscle exercise equipment, which is worn on the penis at normal times such that the pressing amount on the penis is adjusted by the rolling or the unrolling of an end or both ends of a ring-inner-diameter-adjustable multi-protrusion ring, and allows the correct use of muscle to be accurately recognized and confirmed through a self-awareness of penile distension caused when the perineal region muscle necessary for only exercise is contracted, thereby inducing perineal region muscle-reinforcing exercise.

### TECHNICAL FIELD

The present invention relates to a pelvic floor muscle exercise device including a multi-ringed, roll-type elastic cylinder with an adjustable internal diameter, and more particularly to a male perineal muscle exercise device, which can be worn on a proximal portion of the penis at any time 24 hours a day and which enables a user to accurately recognize and confirm the correct use of the perineal muscle through realization of penis expansion, while adjusting the pressing force applied to the penis by rolling or unrolling protrusion rings disposed at one or both ends of the exercise device to adjust the inner diameters of the protrusion rings, thereby enabling effective perineal-muscle-strengthening exercise.

### BACKGROUND ART

To date, there has been no intuitive way of enabling exercisers such as adult males and females to accurately recognize a perineal muscle and to directly determine whether they are performing contraction and relaxation exercises properly in order to strengthen the muscle.

Of course, a perineal-muscle-strengthening exercise is known as one of nonpharmacological and nonsurgical therapies that are mainly recommended in women's rehabilitation clinics for the purpose of improving women's urinary incontinence. However, the perineal muscle exercise is very important not only in improving women's urinary incontinence, but also to men.

That is, as a person enters middle age and the body ages, erectile dysfunction, post-micturition dribbling (a phenomenon in which urine drops on underpants after urination), and weak ejaculation (a phenomenon in which semen ejaculation power is lowered) occur increasingly due to cardiovascular and muscular weakness. These phenomena may be caused by abdominal obesity, working conditions in which a worker is required to carry heavy things or stand for a long time, or rarely, perineal disease. Most of all, however, the main cause is the weakening of perineal muscle with aging, which no one can avoid.

Therefore, urologists, who professionally treat urinary disorder, recommend perineal-muscle-strengthening exercise to men in order to slow or alleviate the above phenomena. This exercise, also known as a Kegel exercise, is performed to thicken the perineal muscle and increase muscular power by intentionally repeating contraction and relaxation of the perineal muscle according to a certain number of times, an interval, a time period and constriction power, which are set in advance, once or several times every day or even during certain activities.

Among the various muscle layers of the perineum, the muscles that are most involved in male sexual function (such as erection, ejaculation, etc.) and urination function and that absolutely need to be strengthened are the bulbocavernosus muscle and the ischiocavernosus muscle, which are located at the front side of superficial transverse perineal muscle.

However, in the case in which an exerciser exercises without accurately recognizing this important specific perineal muscle, the wrong muscle (such as a lower abdominal, hip or thigh muscle) may be used, which may increase abdominal pressure and may thus worsen the original symptoms or function as the cause of such symptoms. Thus, attention is required.

Therefore, it is very important to receive formal training from an expert before and after the start of exercise and to check whether the exercise is being accurately performed using the right muscles. Of course, a patient may visit a urology clinic to use specialized medical equipment for strengthening the perineal muscle using an electromagnetic field or may visit an electromyogram laboratory with a specialized medical personnel to get an expensive medical examination in which a plurality of electrodes is attached to the perineum and surrounding muscles and a neuromuscular state is analyzed by comparing medical examination values and graphs. However, specialized medical personnel and equipment are required for examination and analysis, and thus it is inefficient in terms of time and expense for ordinary persons, who spend most of their time in the workplace, except for extraordinary circumstances where they are in the hospital.

Therefore, trainers specializing in perineal muscle exercise recommend some methods to enable an exerciser to accurately recognize or confirm his perineal muscle by himself. One such recommended method is for the exerciser to lower his pants below his abdomen, repeatedly contract and relax only the perineal muscle, and directly check whether the penis and scrotum are swinging upwards and downwards due to the contraction and relaxation exercises while standing in front of a mirror. In other words, when the penis swings upwards and downwards or when the scrotum shrinks and is lifted toward the body, the exerciser may accurately determine by himself that the perineal muscle is being used. However, only when the exerciser is alone in his room, he can take off his pants and stand in front of a mirror. Thus, it is very difficult for ordinary persons who are restricted by time and space to perform this method in real life.

As described above, perineal muscle exercise is very important and effective to maintain healthy life of all men. However, it is not easy for a trainer (such as a doctor, a nurse, a physiotherapist, an exercise therapist, etc.) or a trainee (such as a patient, an ordinary person who requires exercise, etc.) to find an effective way to check whether the exercise is being successfully performed even after receiving training on how to accurately recognize the muscle and how to do exercise.

In other words, it is most ideal to spend time being treated with a specialized electromyography device or treatment equipment using an electromagnetic field along with an accurate diagnosis from a related medical specialist and to be educated about the correct exercise regime by a professional nurse of a rehabilitation clinic in order to master the exercise regime. To date, however, there has been no effective way to enable the exerciser to accurately recognize the perineal muscle and to directly determine whether they are performing the muscle exercise properly before, during and after the exercise. Thus, there has been a need to develop a safe method or tool that is cheap, convenient and not harmful to the human body and that allows an exerciser to constantly obtain an exercise effect anywhere at any time 24 hours a day while realizing the exercise effect by himself.

In addition, it has been urgently required to develop a perineal muscle exercise (assistance) device that can be instantly adjusted according to the erected or flaccid state (the length and the thickness) of the exerciser's penis, which is different for each person and changes from time to time according to the sexual behavior pattern as well as the psychological environment.

Domestic and foreign products that have been produced and sold to date for the perineal muscle exercise of men are as follows: <1> a cone-shaped heavy metallic weight that is designed to be inserted into the anus in order to increase the constriction power of the anal muscle (https://bestforlife.com/product/mpt/); <2> a product that includes a loop and a weight and that enables a user to train and strengthen muscles around his penis by hanging the weight on his erect penis using the loop and repeatedly lifting the weight by applying force to his penis like weightlifting (refer to http://www.nydailynews.com/life-style/health/men-kegels-new-device-article-1.1869335); <3> a pad-type instrument for obtaining a positive effect by massaging a perineal muscle of men to improve blood flow (http://www.kegelpad.com/KegelPad_Instructions.html); and <4> an electromagnetic chair for female urinary incontinence therapy and similar products (http://www.huffingtonpost.com/entry/kgoal-boost-penis-kegel_us_566861abe4b0f290e521764d). However, the cost of purchasing the above products is high, and a user absolutely needs a separate restricted space and dedicated time to use them.

In addition, sexual devices for men made of various materials and having various shapes, known as “Cock-Rings”, are currently sold. However, these sexual devices are primarily aimed at indulgence in temporary sex play for men who want to satisfy sexual curiosity and who want to stimulate the erogenous zone of a sex partner. However, because these sexual devices have not thoroughly undergone precise anatomical, sexual medical, and clinical medical verifications, they may cause discomfort in actual sex life. In addition, in the case in which men who need different improvements in sexual function or who need effective help depending on their respectively different body states use the above sexual devices while misunderstanding the structure and physiological function of their penis, which frequently changes in rigidity during sexual intercourse, the original discomfort may be increased, or a normal portion or foreskin of the penis may suffer damage to tissue, leading to necrosis of deep tissue.

There have been many actual cases of side effects in using the above sexual devices, including an accident that requires serious treatment such as resection of the penis or skin graft surgery.

However, in some cases, some of these sexual devices are actually used by purchasers in order to prevent slippage of a condom. Some products for preventing condom breakage and slippage are currently produced and sold. However, these products cannot be applied to all different congenital or acquired conditions of the penis and the scrotum. In particular, these products cannot function as an effective condom restrainer that can be flexibly and easily applied to the penis, which frequently and variously changes in rigidity and length during sexual intercourse due to psychological, physical, environmental and psychological factors.

For reference, according to ‘Issues in Sexuality and Sexual Behavior Research: 2013 Edition’, among 1005 men in Croatia between the ages of 18 and 25 in 2010, 18% of 679 men who used condoms experienced condom breakage, and 13% of them experienced condom slippage.

According to another research paper published in 1998, Anthony M A Smith found that a mismatch between the circumference of the penis and the size of the condom was associated with condom breakage (Ref: Does penis size influence condom slippage and breakage? Int J STD AIDS August 1, 1998 9: 444-447).

The “Condoms—a user's guide”, published by Swedish Association for Sexuality Education in 2008, details the causes of condom slippage, damage to a condom film and resultant semen leakage during sexual intercourse. In detail, the length of the penis of 75% of men usually ranges from 12 to 18 cm, with an average of 13 to 15 cm. However, the length of the commercially available condom is 18 cm, which is longer and wider than the average penis, and thus the penis is more likely to escape from the condom by itself. Of course, when a user is not proficient or lacks experience in wearing a condom or uses an excessive amount of lubricant when wearing a condom, it may also be the cause of condom slippage. In addition, in the case in which the penis shaft is inherently thin or in the case in which the penis has the shape of a cone that has a small glans and a thick proximal end, it is also difficult to properly fix the condom on the penis. In addition, in the case of penis anomaly in which the penis is bent, it is not easy to properly put the condom on the penis.

Some opinions of the present applicant, who is also a urologist and an inventor, will now be added hereto. First, the rigidity or the size of the erect penis frequently and variously changes during sexual intercourse due to psychological, physical, environmental and psychological factors, and thus the circumference and the length of the penis also variously change during sexual intercourse. For example, when the size of the penis is decreased, if the woman's vagina contracts sharply or if one of the two persons tries to change their positions, the condom will very easily come off. In particular, when the erect penis is flaccid suddenly shortly after orgasm during sexual intercourse, even a small movement of one person may likely cause only the penis to be pulled out of the condom, with the condom, having become more slippery and heavier due to the discharged semen, left in the vagina. Second, in the case of a penis that has not been circumcised and has excessive foreskin, when the penis is flaccid after ejaculation, the foreskin, which has been moved so as to reveal the glans, may push the condom covering the foreskin as the foreskin returns to its original position. Finally, in the case in which a synthetic resin condom (a plastic condom) is used because of latex allergy, because the plastic condom is less elastic and less tight-fitting than the latex condom, condom slippage is more likely to occur.

### DISCLOSURE

### Technical Problem

Therefore, the present invention has been made in view of the above problems, and it is an object of the present invention to provide a roll-type elastic cylinder for a perineal muscle exercise device including multiple protrusion rings and enabling adjustment of inner diameters of the rings, in which as an upper or lower protrusion ring is rolled and overlaps an adjacent protrusion ring and a film of the cylinder, the overall thickness of the overlapped rolled protrusion rings gradually increases and the inner diameter of the overlapped rolled protrusion rings decreases simultaneously therewith.

It is another object of the present invention to provide a perineal muscle exercise device including multiple protrusion rings and enabling adjustment of inner diameters of the rings, which helps a user thicken his perineal muscle and increase muscular power by intentionally repeating contraction and relaxation of the perineal muscle.

It is a further object of the present invention to provide a device for fixing and pressurizing an external (condom-type) urethral catheter, which is fitted over a penis to drain urine from a patient who is unconscious or has urinary disorder such as an incontinence, a severe urgency, etc., in order to prevent urine from leaking through a gap between the catheter and the penis.

It is still further object of the present invention to provide a condom slippage prevention device for preventing a condom from slipping off a penis when the erect penis is flaccid after sexual intercourse.

### Technical Solution

The present invention provides a perineal muscle exercise device including multiple protrusion rings and enabling adjustment of inner diameters of the rings, which is formed in the shape of a cylindrical elastic tube and in which a penis insertion hole is formed in the center of a main body so that a male penis can be inserted thereinto, an upper protrusion ring is formed at the upper end of the main body, a lower protrusion ring is formed at the lower end of the main body, and one or more intermediate protrusion rings are additionally formed between the upper protrusion ring and the lower protrusion ring.

### Advantageous Effects

A perineal muscle exercise device including multiple protrusion rings and enabling adjustment of inner diameters of the rings of the present invention can be adjusted in size so as to be suitable for the size of a penis inserted thereinto by rolling an upper or lower protrusion ring so that the overall thickness of the rolled protrusion rings increases and the inner diameter thereof naturally decreases simultaneously therewith.

In addition, the present invention can be flexibly, easily and instantly applied to the penis, which frequently and variously changes in rigidity and length during sexual intercourse due to psychological, physical, environmental and psychological factors.

According to the present invention, either one or both of the upper protrusion ring and the lower protrusion ring are rolled or unrolled so as to adjust the pressing force applied to a penis inserted into the protrusion rings, thereby enabling a user to correctly perform perineal-muscle-strengthening exercise while accurately recognizing his specific perineal muscle, which needs to be strengthened, by realizing penis expansion, which occurs due to contraction of the perineal muscle.

In addition, a perineal muscle exercise device including multiple protrusion rings and enabling adjustment of inner diameters of the rings of the present invention is very useful for fixing an external (condom-type) urethral catheter, which is reusable for several days, in an intensive care unit or nursing facility, in which a disposable external (condom-type) urethral catheter must be fitted over a penis of a patient with urinary disorder every day and another device must also be prepared in order to prevent urine from leaking through a gap between the external urethral catheter and the penis whenever a new catheter is fitted over the penis. In addition, the present invention is very useful for fixing a condom or minimizing leakage of discharged semen so as to avoid undesired pregnancy attributable to a condom slippage phenomenon in which, when an erect penis is flaccid suddenly shortly after orgasm during sexual intercourse, only the penis is pulled out of the condom, and the condom, having become more slippery and heavier due to the discharged semen, remains in the vagina.

### DESCRIPTION OF DRAWINGS

FIG. 1 is a perspective view of a perineal muscle exercise device including multiple protrusion rings and enabling adjustment of inner diameters of the rings according to the present invention.

FIG. 2 is a side view of the perineal muscle exercise device including multiple protrusion rings and enabling adjustment of inner diameters of the rings according to the present invention.

FIG. 3 is a perspective view of the perineal muscle exercise device including multiple protrusion rings and enabling adjustment of inner diameters of the rings according to the present invention.

FIG. 4 shows an exploded side view and a sectional view of the perineal muscle exercise device including multiple protrusion rings and enabling adjustment of inner diameters of the rings according to the present invention.

FIG. 5 shows an exploded side view and a sectional view of the perineal muscle exercise device including multiple protrusion rings and enabling adjustment of inner diameters of the rings according to the present invention.

FIG. 6 shows an exploded side view and a sectional view of the perineal muscle exercise device including multiple protrusion rings and enabling adjustment of inner diameters of the rings according to the present invention.

FIG. 7 is a perspective view of the perineal muscle exercise device including multiple protrusion rings and enabling adjustment of inner diameters of the rings according to the present invention.

FIG. 8 is an exploded sectional view of the perineal muscle exercise device including multiple protrusion rings and enabling adjustment of inner diameters of the rings according to the present invention.

FIG. 9 is a view showing an example to which the perineal muscle exercise device including multiple protrusion rings and enabling adjustment of inner diameters of the rings according to the present invention is applied.

FIG. 10 is a view showing an example to which the perineal muscle exercise device including multiple protrusion rings and enabling adjustment of inner diameters of the rings according to the present invention is applied.

FIG. 11 is a side view of the perineal muscle exercise device including multiple protrusion rings and enabling adjustment of inner diameters of the rings according to the present invention.

FIG. 12 is a side view of the perineal muscle exercise device including multiple protrusion rings and enabling adjustment of inner diameters of the rings according to the present invention.

FIG. 13 is a side view of the perineal muscle exercise device including multiple protrusion rings and enabling adjustment of inner diameters of the rings according to the present invention.

FIG. 14 is a view showing an example to which the perineal muscle exercise device including multiple protrusion rings and enabling adjustment of inner diameters of the rings according to the present invention is applied.

FIG. 15 is an anatomical chart showing a bulbocavernosus muscle and an ischiocavernosus muscle of a male penis perineum.

### BEST MODE

Hereinafter, a perineal muscle exercise device including multiple protrusion rings and enabling adjustment of inner diameters of the rings according to preferred embodiments of the present invention will be described in detail with reference to the accompanying drawings.

It is to be understood that the phraseology or terminology used in the following specification and appended claims should not be construed as being limited to general and dictionary meanings but should be construed as having meanings and concepts according to the spirit of the present invention on the basis of the principle that the inventor is permitted to define appropriate terms for the best explanation.

The preferred embodiments described in the following specification and shown in the accompanying drawings are illustrative only and are not intended to represent all aspects of the invention, and thus it is to be understood that various equivalents and modifications can be made without departing from the spirit of the invention.

The present invention relates to a perineal muscle exercise device including multiple protrusion rings and enabling adjustment of inner diameters of the rings, which is formed in the shape of a cylindrical elastic tube including multiple protrusion rings so that a male penis can be inserted thereinto. The device includes a penis insertion hole 12 formed in the center of a main body, an upper protrusion ring 13 formed at the upper end of the main body, a lower protrusion ring 14 formed at the lower end of the main body, one or more intermediate protrusion rings 15 and 16 formed between the upper protrusion ring 13 and the lower protrusion ring 14, and a film ring 11 formed between adjacent ones of the protrusion rings. Either one or both of the upper protrusion ring 13 and the lower protrusion ring 14 are rolled or unrolled so as to adjust the degree to which the penis is pressed by the inner sides of the rings according to the overall thickness of the rolled rings, thereby enabling a user to do exercise while accurately and easily recognizing and confirming the bulbocavernosus muscle and the ischiocavernosus muscle of the penis perineum, which needs to be strengthened, by realizing penis expansion, which occurs due to contraction of the bulbocavernosus muscle and the ischiocavernosus muscle of the perineum.

The perineal muscle exercise device including multiple protrusion rings and enabling adjustment of inner diameters of the rings of the present invention is characterized in that it is made of any elastic material such as latex, polyurethane, silicone, etc.

The perineal muscle exercise device including multiple protrusion rings and enabling adjustment of inner diameters of the rings of the present invention is characterized in that the thickness of the upper protrusion ring 13 is 2 to 8 times the thickness of the intermediate protrusion rings 15 and 16.

The perineal muscle exercise device including multiple protrusion rings and enabling adjustment of inner diameters of the rings of the present invention is characterized in that the thickness of each ring gradually increases from the lower protrusion ring 14 to the upper protrusion ring 13.

The perineal muscle exercise device including multiple protrusion rings and enabling adjustment of inner diameters of the rings of the present invention is characterized in that the thickness of the lower protrusion ring 14 is 2 to 8 times the thickness of each of the intermediate protrusion rings 15 and 16.

The perineal muscle exercise device including multiple protrusion rings and enabling adjustment of inner diameters of the rings of the present invention is characterized in that the upper protrusion ring 13 is rolled toward the lower protrusion ring 14 or in that the lower protrusion ring 14 is rolled toward the upper protrusion ring 13, with the result that the overall thickness of the rolled rings naturally increases and the inner diameter of the rolled rings decreases simultaneously therewith.

The perineal muscle exercise device including multiple protrusion rings and enabling adjustment of inner diameters of the rings of the present invention is characterized in that the inner diameters of the upper protrusion ring 13 and the lower protrusion ring 14 are set such that when a male penis 100 is inserted into the device of the invention, the upper protrusion ring 13 and the lower protrusion ring 14 are caught by a glans corona 111 in order to prevent the device of the invention from slipping to the tip of a penis glans 110.

The perineal muscle exercise device including multiple protrusion rings and enabling adjustment of inner diameters of the rings of the present invention is characterized in that a plurality of through-holes 20 is formed in a mesh shape in the film ring 11 formed between adjacent ones of the protrusion rings.

The perineal muscle exercise device including multiple protrusion rings and enabling adjustment of inner diameters of the rings of the present invention is characterized in that a plurality of anti-slippage projections 30 is formed on the inner surface or the outer surface of the film ring 11.

The perineal muscle exercise device including multiple protrusion rings and enabling adjustment of inner diameters of the rings of the present invention is characterized in that a condom 200 is worn on the inner surfaces or the outer surfaces of the protrusion rings.

The perineal muscle exercise device including multiple protrusion rings and enabling adjustment of inner diameters of the rings of the present invention is characterized in that it is used when an external condom-type urethral catheter 300 is fitted over the penis to drain urine from a patient who is unconscious or has urinary disorder.

The perineal muscle exercise device including multiple protrusion rings and enabling adjustment of inner diameters of the rings of the present invention is characterized in that the protrusion rings contain a fluorescent or luminous material for facilitating nighttime use.

The present invention is characterized in that the protrusion rings contain a pigment or perfume material for motivating a user to perform muscle-strengthening exercise. The present invention is further characterized in that a sensor 50 is mounted therein in order to remotely control the exercise state. The present invention is further characterized in that a vibrator 60 is mounted therein.

The present invention is characterized in that a sound-generating element or a small bell 70 is mounted in the protrusion rings in order to motivate a user to perform muscle-strengthening exercise.

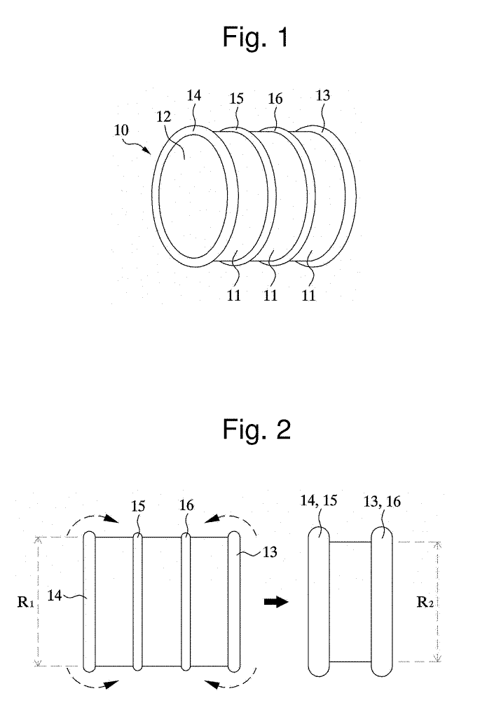

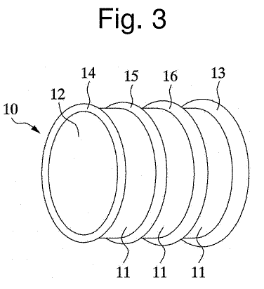

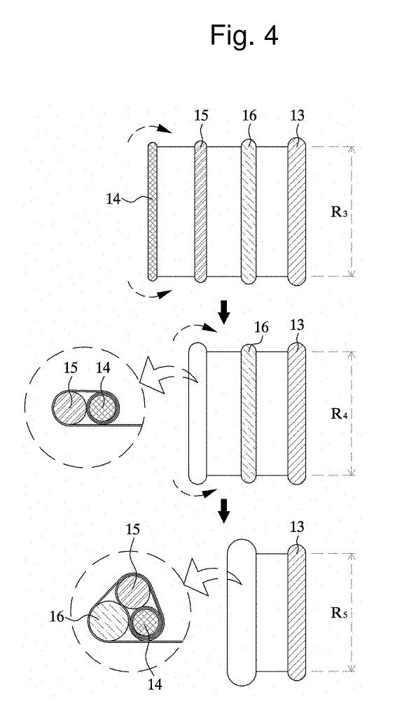

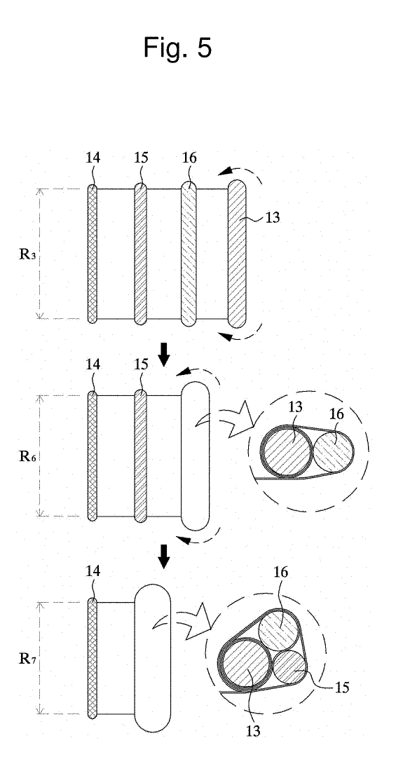

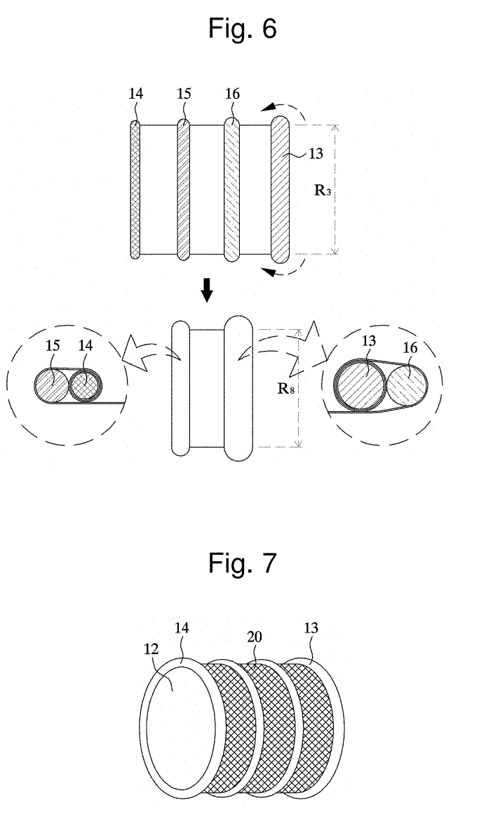

As shown in FIGS. 1 to 6, the present invention is characterized in that, as the multiple protrusion rings and the film ring are rolled together and overlap each other, the overall thickness of the rolled rings increases and in that the protrusion rings and the film ring, which have been rolled into one body, are supported by a neighboring protrusion ring that is not yet rolled, with the result that the rolled state is securely maintained and consequently the rings are prevented from being easily unrolled.

As shown in FIGS. 1 to 6, the present invention has a length of 2 to 10 cm, preferably 4 to 5 cm, and an inner diameter of 2 to 9 cm, preferably 4 to 4.5 cm, depending on the congenital penis size and the expansion thereof.

As shown in FIGS. 1 to 6, the present invention is configured such that the penis insertion hole 12 is formed in the center of the cylindrical-shaped main body so that the male penis can be inserted thereinto, such that the upper protrusion ring 13 is formed at the upper end of the main body so as to have a thickness that is 2 to 8 times the thickness of each of the intermediate protrusion rings 15 and 16, such that the lower protrusion ring 14 is formed at the lower end of the main body so as to have a thickness that is 2 to 8 times the thickness of each of the intermediate protrusion rings 15 and 16, and such that one or more intermediate protrusion rings 15 and 16 are formed between the upper protrusion ring 13 and the lower protrusion ring 14 so that each of them has a thickness that is less or more than the thickness of each of the upper protrusion ring 13 and the lower protrusion ring 14.

The present invention is made of any elastic material such as latex, polyurethane, silicone, etc., and is preferably made of a material having excellent stretchability.

As shown in FIGS. 1 to 6, according to the present invention, as the upper protrusion ring 13 is rolled toward the lower protrusion ring 14 or as the lower protrusion ring 14 is rolled toward the upper protrusion ring 13, the overall thickness of the rolled protrusion rings naturally increases, and the inner diameter of the rolled protrusion rings gradually decreases from R₁ to R₂ or to R₃ simultaneously therewith.

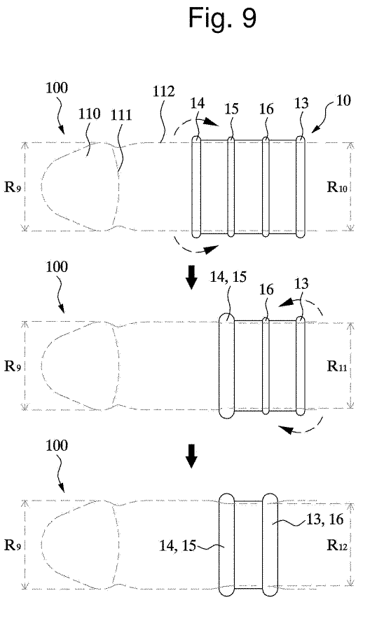

As shown in FIG. 9, according to the present invention, once the upper end and the lower end of the main body and all of the protrusion rings 13, 14, 15 and 16 formed therebetween are fitted over the male penis 100, the penis is not easily pulled out of the device of the invention because the device is made of an elastic material that can be adapted to the circumference of the penis, which varies depending on whether the penis is in an erect or flaccid state. Even after ejaculation during sexual intercourse, the multiple protrusion rings, which have been rolled and thus thickened, are caught by the glans corona 111 of the penis, and thus the device of the invention is prevented from slipping to the tip of the penis glans 110. In addition, the upper protrusion ring 14 has an inner diameter that is set so as to prevent the device of the invention from easily slipping off the male penis 100.

As shown in FIG. 7, the present invention is characterized in that a plurality of concave-convex portions or through-holes 20 is formed in a mesh shape in the film ring 11 formed between adjacent ones of the protrusion rings and in that the size or the number of through-holes 20 may be set high so as to facilitate ventilation between the exercise device of the invention and the penis skin, thereby protecting the skin.

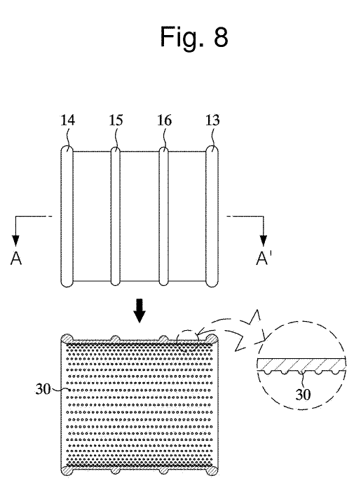

As shown in FIG. 8, the present invention is characterized in that a plurality of anti-slippage projections 30 is formed on the inner surface of the film ring 11 formed between adjacent ones of the protrusion rings in order to perform acupressure on the penis as well as to prevent the exercise device of the invention fitted over the penis from easily slipping to the front or rear end of the penis. In addition, the present invention preferably includes a plurality of projections formed on both the inner surface and the outer surface thereof.

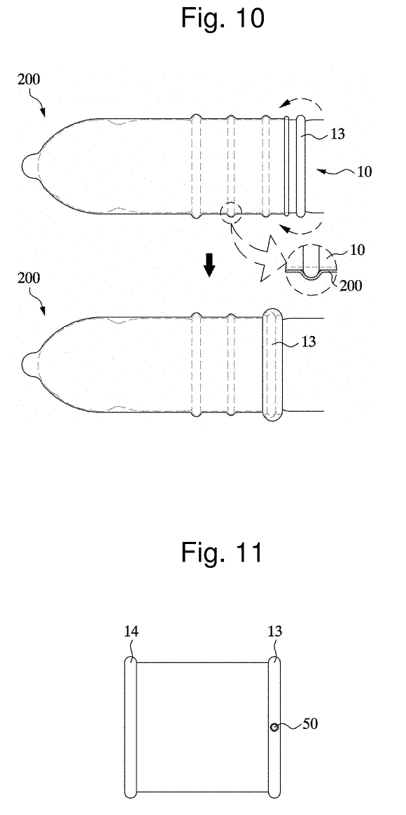

As shown in FIG. 10, the condom 200 is preferably worn on the inner surfaces or the outer surfaces of the protrusion rings of the invention.

The present invention enables a user to perform perineal-muscle-strengthening exercise while accurately recognizing and confirming the specific perineal muscle anywhere at any time 24 hours a day whenever the user desires, helps use a condom safely in any penis state, and can be reused.

The present invention is preferably made of latex or polyurethane, which is highly elastic and stretchable. Compared to latex, polyurethane rolls well at both ends thereof and is generally highly elastic, but is weak and soft.

In the present invention, the upper and lower protrusion rings 13 and 14, which are rolled easily, are formed at both ends of the main body, a plurality of intermediate protrusion rings 15 and 16 having various thicknesses and spaced apart from each other at various intervals are formed between the upper and lower protrusion rings 13 and 14, and the thin mesh-shaped film ring 11 connecting the rings at intervals set according to the thicknesses of the rings are formed between adjacent ones of the protrusion rings. In addition, in the present invention, each of the rings may be formed to have a circular cross-sectional shape or a non-circular cross-sectional shape, such as a polygonal shape.

The material of the present invention is preferably silicon, which has higher shape-retaining ability and strength but is thicker and less elastic than latex or polyurethane. In the case of silicon, when one or both ends of the main body are rolled, the intermediate protrusion rings may function to maintain the rolled state, and the mesh-shaped film ring may be formed between adjacent ones of the rings in order to connect the same. In addition, each of the rings may be formed to have a circular cross-sectional shape or a non-circular cross-sectional shape such as a polygonal shape depending on the circumstances. The material and the shape of the present invention may vary depending on the circumstances.

The device of the present invention is preferably configured such that the protrusion rings contain a fluorescent or luminous material for facilitating nighttime use and also contain a pigment or perfume material for motivating a user to perform muscle-strengthening exercise.

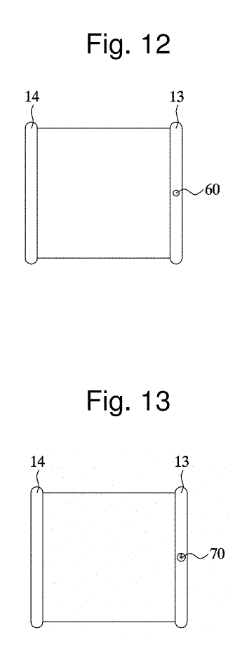

As shown in FIG. 11, the present invention preferably includes a sensor 50 mounted therein in order to collect information about the exercise state and to remotely control the exercise state, and, as shown in FIG. 12, the present invention preferably includes a vibrator 60 mounted therein.

As shown in FIG. 13, the present invention is preferably configured such that a sound-generating element or a small bell 70 is mounted in the protrusion rings in order to motivate a user to perform muscle-strengthening exercise.

The present invention contributes to strengthening and maintaining the erection power during sexual intercourse. Every man has a different penis thickness, and the rigidity of the penis varies depending on the man's state of sexual excitement. According to the present invention, when the protrusion rings 13 and 14, which are disposed at one or both ends of the main body, are rolled toward the center or the opposite end, the overall thickness of the rolled protrusion rings increases, and thus the inner diameter of the thickened rolled protrusion rings naturally decreases from R₁ to R₂, as shown in FIG. 2. In this manner, the pressing force applied to the inside of the multiple protrusion rings may be adjusted merely by rolling the protrusion rings 13 and 14.

In the case in which a user intends to improve and strengthen his weakened erection power through the perineal-muscle-strengthening exercise, the user can arbitrarily adjust the pressing force that is transmitted to the corpus cavernosum penis in stages (multi-stage adjustment is possible) merely by rolling or unrolling the protrusion rings disposed at one or both ends of the device of the invention according to his intention and purpose and changes in the state of erection of the penis. Specifically, the blood introduced through the deep artery due to sexual excitement and stimulation may be retained, and the outflow of the blood from the corpus cavernosum penis through the superficial veins may be appropriately suppressed. As a result, during actual sexual intercourse, it is possible to further strengthen erection ability and to further increase the erection time through a convenient and safe method that can be performed appropriately according to the user's penis state or a change in the penis rigidity.

In addition, the present invention may also be used as an auxiliary device for confirming and maintaining the effect of the perineal muscle exercise anywhere for improving erection power and ejaculation power in daily life. Since the device of the invention is formed to have a diameter for preventing slippage to the tip of the penis glans, a user can wear the device of the invention on his penis, which is usually in a flaccid state. When a user wishes to perform perineal muscle exercise at a desired time and place at any time, the user can accurately recognize and confirm the correct use of the perineal muscle through realization of penis expansion while adjusting the pressing force applied to the penis by rolling or unrolling the protrusion rings disposed at one end (or both ends) of the device of the invention.

In addition, in the case in which a condom is used for contraception or prevention of venereal disease during sexual intercourse, the present invention functions to prevent the condom from being damaged (torn). A commercially available condom having a uniform size does not exactly match the size (the length and the thickness) of the penis of each person, and thus there is a risk of leakage of semen attributable to slippage and separation of the condom or damage to the film at any time during or after sexual intercourse. However, when the condom is worn on the multiple protrusion rings of the invention, which have already been put on the penis, or when the multiple protrusion rings of the invention are worn on the condom, which has already been put on the penis, the condom ring is positioned in the middle of the multiple protrusion rings, and is wrapped and rolled together with the multiple protrusion rings toward one end of the device of the invention. In this way, as the multiple protrusion rings and the condom ring are rolled together, the penis is appropriately pressed, and the condom ring is fixed at a desired position. Therefore, it is possible to minimize a phenomenon in which the condom moves due to loose fitting thereof or in which the film is folded and thus the condom is torn or damaged. In addition, the multiple protrusion rings of the present invention adjust the pressing force that is applied to the penis, thereby facilitating the inflow of blood into the corpus cavernosum penis, suppressing the outflow of blood therefrom, and consequently improving the erection rigidity.

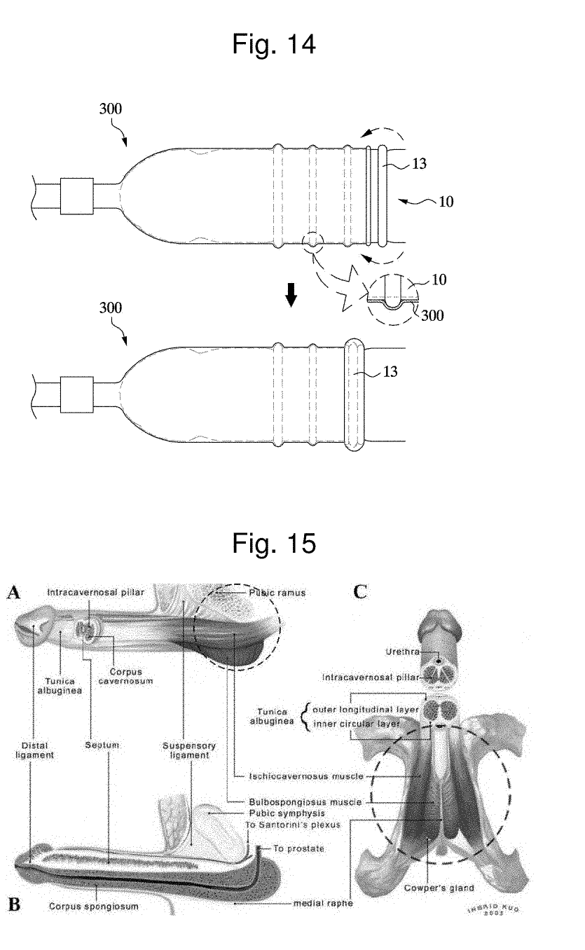

In addition, as shown in FIG. 14, when an external (condom-type) urethral catheter 300 is fitted over the penis to drain urine from a patient who is unconscious or has urinary disorder or when a condom is used for contraception or prevention of venereal disease during sexual intercourse, the condom or the external (condom-type) urethral catheter is fixed and is prevented from slipping off the penis by the present invention.

Since a commercially available condom has a uniform size and is sold to various members of the public, each of whom has a penis of a different size, there is a risk of pregnancy by leakage of semen into a vagina attributable to slippage and separation of the condom from the penis or damage to the film during or after sexual intercourse.

However, when the condom is worn on the device of the invention, which has already been put on the penis, or when the device of the invention is worn on the condom, which has already been put on the penis, the condom ring is positioned in the middle of the multiple protrusion rings, and is wrapped and rolled together with the multiple protrusion rings toward one end of the device of the invention. This helps improvement of erection rigidity by facilitating the inflow of blood into the corpus cavernosum penis and suppressing the outflow of blood therefrom, and also fixes the condom at a desired position. Therefore, it is possible to minimize a phenomenon in which the condom slips off the penis during reciprocating movement of the penis in the vagina or in which the condom, having become slippery by semen, slips off the penis after ejaculation. Even when the condom slips slightly from the penis, the rolled portion of the device of the invention tightly holds the entrance portion of the condom, and thus the condom ring is caught by the glans corona, thereby preventing the condom from completely slipping off the penis and also minimizing leakage of the discharged semen, collected in the condom, outside the condom.

The present invention is a perineal-muscle-strengthening exercise device. All of the muscles of the perineum play an important role. Particularly, among them, the bulbocavernosus muscle and the ischiocavernosus muscle are muscles that wrap more than half the proximal portion of the penis in both lower sides of the proximal portion of the penis. These muscles function to support the penis during erection as well as to push blood from the proximal portion of the penis to the distal portion of the penis, and thus play an important role in the erection mechanism.

In other words, if a user receives sexual stimulus in an audio-visual or psychological way while performing exercise of contracting the bulbocavernosus muscle and the ischiocavernosus muscle, the amount of blood flowing from the proximal portion of the corpus cavernosum penis to the distal portion thereof increases, with the result that the time taken for penis erection is shortened, and erection power is further increased. Further, if a user performs bulbocavernosus muscle contraction exercise and receives psychological (audio-visual) stimulus at the same time while wearing the exercise device of the invention, the amount of blood flowing into the corpus cavernosum penis is increased and the outflow of blood therefrom is suppressed to a certain extent by the contraction of the bulbocavernosus muscle. Accordingly, the user is capable of immediately experiencing erection visually or sensually, obtaining an increase in erection power, and checking whether a Kegel exercise is being correctly performed.

A user can wear or use the present invention regardless of time and place. It is recommended to perform perineal muscle exercise continuously and periodically (more than three times a day) in a planned session and method anywhere and at any time for any clinical purpose. The present invention enables ordinary persons to perform perineal muscle exercise safely and continuously while checking whether the exercise is being correctly performed merely by learning how to use the present invention, thereby alleviating symptoms of discomfort and enhancing sexual function.

Among men between the ages of 24 and 75 who have visited the clinic operated by the present applicant, who is also an inventor, during about 18 months from 2013 to 2015, 87 men (Group A: 14 healthy men who did not complain of any symptoms, Group B: 25 men who complained of a post-micturition dribbling symptom, Group C: 21 men who complained of a slight erectile dysfunction symptom, Group D: 15 men who complained of a weak ejaculation symptom, and Group E: 12 men who complained of both an erectile dysfunction symptom and a weak ejaculation symptom) who did not need to immediately take additional specialized medical examinations or to be treated with specialized medicine participated in an experiment, and the present applicant observed the effects of the perineal muscle exercise device 10. Specifically, the participants answered the International Index of Erectile Function (IIEF) questionnaire and the questionnaire of the clinic, were subjected to a physical examination of the urinary system, and learned a self-training method for performing perineal-muscle-strengthening exercise. Then, the present applicant compared and analyzed the effects of the exercise between a non-user group, in which the members did perineal-muscle-strengthening exercise three times a day for three weeks without using the exercise device of the present invention, and a user group, in which the members did perineal-muscle-strengthening exercise three times a day for three weeks using the exercise device of the present invention.

In the non-user group, 3 out of 4 (75%) in healthy Group A, 7 out of 12 (58%) in Group B with a post-micturition dribbling symptom, 5 out of 10 (50%) in Group C with a slight erectile dysfunction symptom, 5 out of 9 (55%) in Group D with a weak ejaculation symptom, and 4 out of 7 (57%) in Group E with slight erectile dysfunction and weak ejaculation symptoms experienced meaningful improvement after exercise.

On the other hand, in the user group that used the exercise device of the present invention, 9 out of 10 (90%) in healthy Group A, 11 out of 13 (84%) in Group B with a post-micturition dribbling symptom, 9 out of 11 (81%) in Group C with a slight erectile dysfunction symptom, 5 out of 6 (83%) in Group D with a weak ejaculation symptom, and 4 out of 5 (80%) in Group E with slight erectile dysfunction and weak ejaculation symptoms experienced meaningful improvement after exercise. As described above, about 50% to 75% (an average of 59%) of the members in the group that did not use the exercise device of the present invention experienced improvement, whereas about 80% to 92% (an average of 85%) of the members in the group that used the exercise device of the present invention experienced much greater improvement. With the global increase in the elderly population, the exercise device of the present invention is expected to increasingly contribute to improvement in the urinary system as well as the personal hygiene of elderly men all over the world by helping them correctly perform perineal-muscle-strengthening exercise while accurately recognizing the perineal muscle.

TABLE 1
Comparison test result between a user group that used the exercise device of the present invention
and a non-user group that did not use the exercise device of the present invention.

| | | Non-user Group | | | User Group | | |
|---|---|---|---|---|---|---|---|
| | | | Number of | | | Number of | |
| | Total | | Participants | | | Participants | |
| | Number of | Number of | Showing | Improvement | Number of | Showing | Improvement |
| Class. | Participants | Participants | Improvement | (%) | Participants | Improvement | (%) |
| Group A | 14 | 4 | 3 | 75 | 10 | 9 | 90 |
| Group B | 25 | 12 | 7 | 58 | 13 | 12 | 92 |
| Group C | 21 | 10 | 5 | 50 | 11 | 9 | 81 |
| Group D | 15 | 9 | 5 | 56 | 6 | 5 | 83 |
| Group E | 12 | 7 | 4 | 57 | 5 | 4 | 80 |
| Total | 87 | 42 | 24 | 59% | 45 | 39 | 85% |
| | | | | (Average) | | | (Average) |

Group A: healthy men who did not complain of any symptoms, Group B: men who complained of a post-micturition dribbling symptom, Group C: men who complained of a slight erectile dysfunction symptom, Group D: men who complained of a weak ejaculation symptom, and Group E: men who complained of both an erectile dysfunction symptom and a weak ejaculation symptom

When a user performs bulbocavernosus muscle contraction exercise while wearing the exercise device of the invention, the amount of blood flowing into the corpus cavernosum penis is increased and the outflow of blood therefrom is suppressed to a certain extent, with the result that the user can experience penis expansion. Accordingly, the user can perform perineal muscle exercise safely and continuously while checking whether the exercise is being correctly performed anywhere and at any time regardless of time and place.

The present invention motivates men who suffer from psychogenic erectile dysfunction attributable to lack of confidence or venous erectile dysfunction attributable to aging or patients who suffer from urinary disorder such as irritable bladder or post-micturition dribbling to perform exercise for improving urination function and sexual function, thereby reducing unnecessary medical treatment and side effects and economic loss attributable thereto.

Psychogenic erectile dysfunction, which is not associated with age, or weakening of the rigidity of an erect penis attributable to aging may happen to anyone at any time. Safe and specialized treatment for improving penis rigidity without any additional expenses is perineal-muscle-strengthening exercise. In other words, due to the perineal muscle exercise as well as a tourniquet effect in which the blood introduced through the deep artery is retained and the outflow of the blood from the corpus cavernosum penis through the superficial veins is suppressed when the penis is inserted into or moves reciprocatingly in the vagina during actual sexual intercourse, sexual intercourse, with the proximal portion of the penis pressed appropriately by the device of the present invention, may help improve erectile dysfunction to a certain extent, like the principle of a vacuum pump or a constriction device used for treating patients who suffer from erectile dysfunction.

1. A perineal muscle exercise device including multiple protrusion rings and enabling adjustment of inner diameters of the rings, wherein the device is formed in a shape of a cylindrical elastic tube including multiple protrusion rings so that a male penis is inserted thereinto, a penis insertion hole is formed in a center of a main body, an upper protrusion ring is formed at an upper end of the main body, a lower protrusion ring is formed at a lower end of the main body, one or more intermediate protrusion rings are formed between the upper protrusion ring and the lower protrusion ring, a film ring is formed between adjacent ones of the protrusion rings, and either one or both of the upper protrusion ring and the lower protrusion ring are rolled or unrolled so as to adjust a degree to which a penis is pressed by inner sides of the rings according to an overall thickness of the rolled rings, thereby enabling a user to do exercise while accurately and easily recognizing and confirming a bulbocavernosus muscle and an ischiocavernosus muscle of a penis perineum, which needs to be strengthened, by realizing penis expansion that occurs due to contraction of the bulbocavernosus muscle and the ischiocavernosus muscle of the penis perineum.

2. The perineal muscle exercise device according to claim 1, wherein the device is made of any one material selected from among latex, polyurethane and silicone.

3. The perineal muscle exercise device according to claim 1, wherein a thickness of each of the rings gradually increases from a one (15) of the intermediate protrusion rings that is located near the lower protrusion ring (14) to the upper protrusion ring (13).

4. The perineal muscle exercise device according to claim 1, wherein a thickness of the upper protrusion ring (13) is 2 to 8 times a thickness of each of the intermediate protrusion rings (15 and 16).

5. The perineal muscle exercise device according to claim 1, wherein a thickness of the lower protrusion ring (14) is 2 to 8 times a thickness of each of the intermediate protrusion rings (15 and 16).

6. The perineal muscle exercise device according to claim 1, wherein the upper protrusion ring (13) is rolled toward the lower protrusion ring (14) or the lower protrusion ring (14) is rolled toward the upper protrusion ring (13), whereby an overall thickness of the rolled rings naturally increases and an inner diameter of the rolled rings decreases simultaneously therewith.

7. The perineal muscle exercise device according to claim 1, wherein inner diameters of the upper protrusion ring (13) and the lower protrusion ring (14) are set such that when a male penis (100) is inserted into the device, the upper protrusion ring (13) and the lower protrusion ring (14) are caught by a glans corona (111) of the male penis in order to prevent the device from slipping to a tip of a glans (110) of the male penis.

8. The perineal muscle exercise device according to claim 1, wherein a plurality of through-holes (20) is formed in a mesh shape in the film ring (11) formed between adjacent ones of the protrusion rings.

9. The perineal muscle exercise device according to claim 1, wherein a plurality of anti-slippage projections (30) is formed on an inner surface or an outer surface of the film ring (11).

10. The perineal muscle exercise device according to claim 1, wherein a condom (200) is worn on inner surfaces or outer surfaces of the protrusion rings.

11. The perineal muscle exercise device according to claim 1, wherein the device is used when an external condom-type urethral catheter (300) is fitted over a penis to drain urine from a patient who is unconscious or has urinary disorder.

12. The perineal muscle exercise device according to claim 1, wherein a fluorescent or luminous material for facilitating nighttime use is contained in the protrusion rings.

13. The perineal muscle exercise device according to claim 1, wherein a pigment or perfume material for motivating a user to perform muscle-strengthening exercise is contained in the protrusion rings.

14. The perineal muscle exercise device according to claim 1, comprising a sensor (50) mounted therein in order to remotely control an exercise state.

15. The perineal muscle exercise device according to claim 1, comprising a vibrator (60) mounted therein.

16. The perineal muscle exercise device according to claim 1, wherein a sound-generating element or a small bell (70) is mounted in the protrusion rings in order to motivate a user to perform muscle-strengthening exercise.
